(19) United States
(12) Patent Application Publication
(10) Pub. No.: US 2026/0338778 A1
(43) Pub. Date: Oct. 29, 2026

(54) Identification of novel biomarkers for breast cancer CTC characterization

(71) Applicant: ScreenCell S.A., Paris (FR)

(72) Inventor: Sina NASERIAN, Saint Maur Des Fosses (FR)

(21) Appl. No.: 19/485,928

(22) PCT Filed: May 21, 2024
(86) PCT No.: PCT/IB2024/000289

§ 371 Date: Nov. 19, 2025

Related U.S. Application Data
(60) Provisional application No. 63/468,041, filed on May 22, 2023.

(51) Int. Cl. G01N 33/57515; C12Q 1/6886

(52) U.S. Cl. CPC G01N 33/57515; C12Q 1/6886; C12Q 2600/158; G01N 2800/7028

(57) ABSTRACT

Provided herein are methods for identifying breast cancer in a subject, the methods comprising providing a biological sample from the subject and determining a differential level of at least one biomarker, wherein the differential level of the at least one biomarker compared to a healthy control or a reference value is indicative for the presence of breast cancer in the subject.

### CROSS REFERENCE

This application claims the benefit of U.S. Provisional Patent Application No. 63/468,041, filed on May 22, 2023, which is incorporated by reference in its entirety.

### BACKGROUND

According to the World Health Organization (WHO), breast cancer is the most common malignant neoplasm in women and is one of the leading causes of cancer deaths in general. Breast cancer is a highly heterogeneous disease, classified into five stages based on molecular expression profiles: (I) luminal (A or B); (II) basal-like estrogen receptor (ER)-negative, progesterone receptor (PR)-negative, and human epidermal growth factor receptor 2 (EGFR2) negative, also called triple-negative breast cancer (TNBC); (III) epidermal growth factor receptor 2 (EGFR2) family expressed (HER2); (IV) normal breast-like (low expression of luminal epithelial genes and high expression of basal epithelial and non-epithelial genes); and (V) claudin-low-expressed breast cancer (low expression of cell-cell junction proteins). Based on their ER status, nearly 70% of breast cancer cases are hormone receptor-positive, 20% of cases overexpress the HER2 oncogene, and 15% of all breast cancers are triple-negative.

Depending on the breast cancer subtype, several prognoses are possible. TNBC has a worse prognosis than luminal A cancer, which has the best prognosis. TNBC is the most aggressive form of breast cancer, with early recurrence, an elevated incidence of visceral metastasis (liver and lung), and a low survival rate, in part because of the lack of response to hormone therapy and of the absence of efficient and personalized medicines.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are methods for identifying breast cancer in a subject, the methods comprising: a) providing a biological sample from the subject; and b) determining a differential level of at least one biomarker of a biomarker set in the biological sample, wherein the differential level of the at least one biomarker of the biomarker set compared to a healthy control or a reference value is indicative for the presence of breast cancer in the subject, and wherein the biomarker set comprises CD55, LSR, MARCKSL1, GPC1, SLC9A3R1, CXADR, SHROOM3, MUC1, PPP1R16A, ATP1B1, RHOD, SYTL2, or any combination thereof. In some embodiments, the biomarker set comprises CD55, LSR, MARCKSL1, GPC1, SLC9A3R1, CXADR, SHROOM3, MUC1, PPP1R16A, ATP1B1, RHOD, and SYTL2. In some embodiments, a differential level of a biomarker selected from CD55, LSR, MARCKSL1, GPC1, SLC9A3R1, CXADR, SHROOM3, MUC1, PPP1R16A, ATP1B1, RHOD, or SYTL2 is a level higher than the healthy control or the reference value. In some embodiments, a differential level of a biomarker selected from CD55, LSR, MARCKSL1, GPC1, SLC9A3R1, CXADR, SHROOM3, MUC1, PPP1R16A, ATP1B1, RHOD, or SYTL2 is a level lower than the healthy control or the reference value. In some embodiments, the method further comprises using RNA sequencing to obtain the differential level of the at least one biomarker. In some embodiments, the method further comprises using one or more antibodies to obtain the differential level of the at least one biomarker. In some embodiments, the method further comprises imaging the biological sample. In some embodiments, the method further comprises using western blot, ELISA, or mass spectrometry to obtain the differential level of the at least one biomarker. In some embodiments, the method further comprises morphological analysis of the biological sample. In some embodiments, the biological sample is a biofluid. In some embodiments, the biofluid is blood, plasma, or serum. In some embodiments, the biofluid is blood. In some embodiments, the sample is obtained prior to the onset of symptoms. In some embodiments, the sample is obtained after the onset of symptoms. In some embodiments, the subject is a human. In some embodiments, there is a kit for performing the methods described herein.

Provided herein are methods for identifying breast cancer in a subject, the methods comprising: a) providing a biological sample from the subject; and b) determining a differential level of at least one biomarker of a biomarker set in the biological sample, wherein an increased level of the at least one biomarker of the biomarker set compared to a healthy control or a reference value is indicative for the presence of breast cancer in the subject, and wherein the biomarker set comprises MARCKSL1 and SLC9A3R1. In some embodiments, the biomarker set further comprises ATP1B1 and RHOD. In some embodiments, the biomarker set comprises MARCKSL1, ATP1B1, and SLC9A3R1. In some embodiments, the biomarker set comprises MARCKSL1, RHOD, and SLC9A3R1. In some embodiments, the biomarker set further comprises LSR. In some embodiments, the biomarker set further comprises CD55, GPC1, CXADR, SHROOM3, MUC1, PPP1R16A, SYTL2, or a combination thereof. In some embodiments, the method further comprises using RNA sequencing to obtain the differential level of the at least one biomarker. In some embodiments, the method further comprises using one or more antibodies to obtain the differential level of the at least one biomarker. In some embodiments, the method further comprises imaging the biological sample. In some embodiments, the method further comprises using western blot, ELISA, or mass spectrometry to obtain the differential level of the at least one biomarker. In some embodiments, the method further comprises morphological analysis of the biological sample. In some embodiments, the biological sample is a biofluid. In some embodiments, the biofluid is blood, plasma, or serum. In some embodiments, the biofluid is blood. In some embodiments, the sample is obtained prior to the onset of symptoms. In some embodiments, the sample is obtained after the onset of symptoms. In some embodiments, the subject is a human. In some embodiments, there is a kit for performing the methods described herein.

Provided herein are methods for identifying breast cancer in a subject, the methods comprising: a) providing a biological sample from the subject; and b) determining a differential level of at least one biomarker of a biomarker set in the biological sample, wherein an increased level of the at least one biomarker of the biomarker set compared to a healthy control or a reference value is indicative for the presence of breast cancer in the subject, and wherein the biomarker set comprises MARCKSL1, SLC9A3R1, and RHOD.

Provided herein are methods for identifying breast cancer in a subject, the methods comprising: a) providing a biological sample from the subject; and b) determining a presence or an absence of at least one biomarker of a biomarker set in the biological sample, wherein the presence or the absence of the at least one biomarker of the biomarker set compared to a healthy control or a reference value is indicative for the presence of breast cancer in the subject, and wherein the biomarker set comprises CD55, LSR, MARCKSL1, GPC1, SLC9A3R1, CXADR, SHROOM3, MUC1, PPP1R16A, ATP1B1, RHOD, SYTL2, or a combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an exemplary bioinformatic workflow for determining biomarkers of interest.

FIG. 2 illustrates an exemplary workflow for gene filtration using Protein Atlas and UniProt databases.

FIG. 3 shows a list of twelve genes with intermediate to high expression levels in breast cancer, and the breast cancer cell lines that express each gene listed.

FIG. 4 illustrates an exemplary in vitro workflow whereby breast cancer cell lines (e.g., MCF7 and SKBR3) are expanded in culture and then 5,000 cells are spiked into 3 mL of healthy blood samples from healthy donors. Circulating tumor cell (CTC) identification techniques are used to filter the blood, and larger cells are retained on the isolation supports. Immunocytochemistry (ICC) methods are used to label leucocytes and breast cancer (BC) cells.

FIG. 5 shows representative images of ICC staining targeting proteins of interest including SLC9A3R1 (Panel A), MARCKSL1 (Panel B), ATP1B1 (Panel C), LSR (Panel D), CD55 (Panel E), and MUC1 (Panel F). Leucocytes and cancer cells (MCF7 or SKBR3) were stained. The cell nucleus was also stained with hematoxylin.

FIG. 6 shows representative images of ICC staining targeting proteins of interest including SHROOM3 (Panel A), PPP1R16A (Panel B), CXADR (Panel C), GPC1 (Panel D), SYTL2 (Panel E), and RHOD (Panel F). Leucocytes and cancer cells (MCF7 or SKBR3) were stained. The cell nucleus was also stained with hematoxylin.

FIG. 7 summarizes the expression profile of each protein.

FIG. 8 shows ICC labeling performed on 20 MCF7 cells spiked into the healthy blood using a SLC9A3RA, MARCKSL1, and ATP1B1 trio cocktail (Panel A) or using a SLC9A3RA, MARCKSL1, and RHOD trio cocktail (Panel B).

FIG. 9 compares the efficacy of control markers (Panel A) and each marker separately and in combinations (Panel B); cocktail 1 is a SLC9A3RA, MARCKSL1, and ATP1B1 trio cocktail; cocktail 2 is a SLC9A3RA, MARCKSL1, and RHOD trio cocktail. The MCF7 cell line was used for all conditions. N=4 for CK and EpCAM conditions; n=3 for all other conditions.

FIG. 10 shows control immunostaining using CD45 and EpCAM markers (Panel A) or CD45 and CK markers (Panel B) on blood samples of breast cancer patients showing a diverse expression with CTCs.

FIG. 11 shows immunostaining using CD45 and ATP1A1 markers on blood samples of five breast cancer patients revealing the absence of ATP1A1 expression by breast cancer CTCs.

FIG. 12 shows immunostaining using CD45 and cocktail 2 (MARCKSL1, SLC9A3R1, and RHOD) on blood samples of eleven breast cancer patients P=patient.

FIG. 13 shows immunostaining using CD45 and cocktail 2 (MARCKSL1, SLC9A3R1, and RHOD) on blood samples of twenty breast cancer patients P=patient.

FIG. 14 shows a computer system that is programmed or otherwise configured to implement the methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Isolation and characterization of circulating tumor cells (CTCs) can be based on common epithelial-specific markers such as epithelial cellular adhesion molecules (EpCAM) and/or cytokeratin (CK). Nevertheless, CTCs of carcinoma origins (cancer that forms in epithelial tissue) have been shown to have a heterogenous expression of EpCAM and CK markers. As a mechanism to improve CTC migratory and invasive properties, these circulating cells usually shift from epithelial to mesenchymal profiles, resulting in a general reduction of epithelial marker expression.

EpCAM is typically downregulated during the epithelial-mesenchymal transition (EMT) in epithelial cancers. This is mediated by extracellular signal-regulated kinases (ERK), EMT transcription factor activity, and other oncogenic mechanisms and pathways. During EMT, EpCAM can also be cleaved during a process called regulated intramembrane proteolysis (RIP), whereby the epithelial cellular intracellular domain (EpICD) is transported into the nucleus and helps to stimulate target gene transcription that promotes growth, cancer stem cell properties and EMT. Additionally, during EMT and in a mechanism that is believed to be a hallmark of this phenomenon, the expression of CK is also downregulated, and inversely, the expression of vimentin (a mesenchymal marker) is upregulated. It has been shown that the expression of CK18 was significantly downregulated in breast cancer (BC) tissues and this was associated with a higher tumor, node, metastasis (TNM) stage, lymph node metastasis, and unfavorable survival in those patients. Moreover, CK18 knockdown is associated with the activation of the NFκB/Snail signaling pathway, which regulates EMT.

Conventional methods for identifying cancers rely on sequestering CTCs with EpCAM and/or CK on their surface, but immunostainings based on these two markers do not identify all existing CTCs, leading to a partial loss of information. The presence of epithelial cells in healthy blood samples occurs and can be related to many factors, such as inflammation. Additionally, the process of blood drawing could cause the entrance of a few skin epithelial cells into the blood samples. Thus, sensitive makers are needed to evaluate this limitation of markers such as EpCAM and CK to isolate identification of the real CTCs that are generated due to the presence of an active tumor.

Methods and systems described herein, have higher sensitivity and accuracy for cancer detection than EpCAM-dependent and/or CK-dependent models, particularly in detection of different kinds of cancers such as head and neck cancer, colorectal cancer, hemangiopericytoma, glioblastomas and breast cancer because methods provided herein can capture additional tumor cells of all sizes and independently of marker expression, while other methods can only capture CTCs that express EpCAM and/or CK on their surface.

### DEFINITIONS

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated, as used herein, the terms “biomarker” or “marker” refers to a biological molecule or characteristic that indicates a normal or abnormal process, condition, or disease. Biomarkers are also known as molecular markers or signature molecules. Biomarkers can be early warning systems for health. A biomarker can indicate susceptibility or risk for a health condition. A biomarker can diagnose a health condition. A biomarker can be used to determine a course of treatment. A biomarker can be used to measure the prognosis of a health condition. A biomarker can be used to monitor a health condition. A biomarker can be used to predict a health condition. A biomarker can be used to measure pharmacodynamic responses. A biomarker can be used to determine safety.

### Circulating Tumor Cells

Provided herein are methods, compositions, and systems for identifying circulating tumor cells (CTCs). CTCs are cells released from primary tumors and transported through the body via blood or lymphatic vessels before settling to form micrometastases under suitable conditions. CTCs can be a negative prognostic factor for survival in cancer (e.g., breast cancer).

Cancer is a disease of tissue growth regulation. For a normal cell to transform into a cancer cell, the genes that regulate cell growth and differentiation must be altered. Affected genes are generally divided into two broad categories. Oncogenes are genes that promote cell growth and reproduction. Tumor suppressor genes are genes that inhibit cell division and survival. Malignant transformation can occur through the inappropriate over-expression of oncogenes or by the inappropriate under-expression or disabling of tumor suppressor genes.

Breast cancer is a type of cancer that develops in breast tissue. Breast cancer is a highly heterogeneous disease, classified into five stages based on molecular expression profiles: (I) luminal (A or B); (II) basal-like estrogen receptor (ER)-negative, progesterone receptor (PR)-negative, and human epidermal growth factor receptor 2 (EGFR2) negative, also called triple-negative breast cancer (TNBC); (III) epidermal growth factor receptor 2 (EGFR2) family expressed (HER2); (IV) normal breast-like (low expression of luminal epithelial genes and high expression of basal epithelial and non-epithelial genes); and (V) claudin-low-expressed breast cancer (low expression of cell-cell junction proteins).

Primary breast cancer tumors can shed circulating breast cancer tumor cells. Identification of breast cancer-related CTCs can be useful for patients with both primary and metastatic disease and can be an important way to both identify patients and to monitor disease progression. Breast cancer CTCs can exist as single cells or as clusters with the latter having significantly more metastatic ability and worse prognosis. In some cases, only a small fraction of CTCs can survive in blood circulation, and even a smaller population can display metastatic potential. Several mechanisms and related factors such as genetic alterations, abnormal gene expression, EMT, stem cell phenotype, shielding effects of platelets, and immune escape mechanisms can be associated with CTC survival. Upregulation of different immunosuppressive molecules such as programmed death-ligand 1 (PDL1) and HLA-G can be part of the breast cancer CTC immune escape mechanism.

Circulating tumor cells can be at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 11 μm, at least about 12 μm, at least about 13 μm, at least about 14 μm, at least about 15 μm, at least about 16 μm, at least about 17 μm, at least about 18 μm, at least about 19 μm, at least about 20 μm, at least about 21 μm, at least about 22 μm, at least about 23 μm, at least about 24 μm, at least about 25 μm, at least about 26 μm, at least about 27 μm, at least about 28 μm, at least about 29 μm, at least about 30 μm, at least about 31 μm, at least about 32 μm, at least about 33 μm, at least about 34 μm, at least about 35 μm, at least about 36 μm, at least about 37 μm, at least about 38 μm, at least about 39 μm, at least about 40 μm, at least about 45 μm, at least about 50 μm, at least about 55 μm, at least about 60 μm, at least about 65 μm, at least about 70 μm, at least about 75 μm, at least about 80 μm, at least about 85 μm, at least about 90 μm, at least about 95 μm, or at least about 100 μm in diameter. Circulating tumor cells can be at most about 100 μm, at most about 95 μm, at most about 90 μm, at most about 85 μm, at most about 80 μm, at most about 75 μm, at most about 70 μm, at most about 65 μm, at most about 60 μm, at most about 55 μm, at most about 50 μm, at most about 45 μm, at most about 40 μm, at most about 39 μm, at most about 38 μm, at most about 37 μm, at most about 36 μm, at most about 35 μm, at most about 34 μm, at most about 33 μm, at most about 32 μm, at most about 31 μm, at most about 30 μm, at most about 29 μm, at most about 28 μm, at most about 27 μm, at most about 26 μm, at most about 25 μm, at most about 24 μm, at most about 23 μm, at most about 22 μm, at most about 21 μm, at most about 20 μm, at most about 19 μm, at most about 18 μm, at most about 17 μm, at most about 16 μm, at most about 15 μm, at most about 14 μm, at most about 13 μm, at most about 12 μm, at most about 11 μm, at most about 10 μm, at most about 9 μm, at most about 8 μm, at most about 7 μm, at most about 6 μm, at most about 5 μm, at most about 4 μm, at most about 3 μm, at most about 2 μm, or at most about 1 μm in diameter. Circulating tumor cells can be larger than other cells (e.g., leukocytes) in a sample (e.g., a blood sample). Circulating tumor cells can be smaller than other cells in a sample (e.g., a blood sample). The size of CTCs can allow them to be filter separated from other types of cells.

### Sample Processing

Provided herein are methods for enriching and identifying CTCs using novel and reliable biomarkers. The biomarkers can consistently and more efficiently detect CTCs among other cells as compared to control methods using housekeeping biomarkers (e.g., CD45) or epithelial-specific biomarkers (e.g., EpCAM, CK).

CTCs can be enriched with high efficiency. CTCs can be enriched by filtering a biofluid sample (e.g., blood) to separate CTCs from other types of cells (e.g., leukocytes). In some embodiments, CTCs are enriched by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more as compared to a control method. In some embodiments, CTCs are enriched by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less as compared to a control method.

Once CTCs are enriched and separated, they can be processed to determine if one or more biomarkers are present or absent in a sample. Alternatively or in addition to, CTCs can be processed to determine a differential level of one or more biomarkers in the sample.

In some embodiments, protein biomarkers are detected and quantified using antibody-based methods. For example, antibodies for specific proteins can be added to a sample and incubated to allow for antibody staining. Antibody-stained samples can then be imaged to determine presence, absence, or amount of a protein in a sample. In some embodiments, quantification of a biomarker comprises quantifying the amount of antibody and/or antigen in the test sample.

In some embodiments, DNA and/or RNA are extracted from a sample. In some embodiments, RNA-sequencing or other sequencing methods can be used to determine the amount of a biomarker in a sample. Housekeeping genes, (e.g., CD45) can be used as controls. In some embodiments, the entire sample is analyzed. Alternatively, single cell analysis can be performed.

A biomarker can be identified as being differentiated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A biomarker can be identified as being differentiated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, the methods provided herein identify at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more CTCs as compared to control methods using epithelial-specific biomarkers (e.g., EpCAM, CK).

In some embodiments, immunostaining is performed in addition to cytological characterization to correctly distinguish CTCs from the rest of the blood cells. In some embodiments about 0.001%, about 0.005%, about 0.01%, about 0.02%, about 0.03%, about 0.04%, or about 0.05% of cells are leucocytes retained on the isolation support following cytological characterization which require additional immunostaining. In some embodiments, about 0.02% of cells are leucocytes retained on the isolation support following cytological characterization which require additional immunostaining. In some embodiments, biomarker analysis alone is used to distinguish CTCs from other cells (e.g., leukocytes).

In some embodiments, additional information, such as biometric parameters, can be used to supplement the biomarker information in determining a breast cancer diagnosis. Additional parameters can include but are not limited to: age, gender, carcinogen exposure history, and family history of breast cancer.

In some embodiments, immunostaining and imaging of samples can be used as a control. Alternatively or in addition to, epithelial markers such as EpCAM and CK can also be used as controls.

Samples provided herein can be biofluids or bodily fluids. Biofluids can include, but are not limited to, blood, serum, urine, mucous, tears, sweat, synovial fluid, plasma, saliva, breast milk, or cerebrospinal fluid.

Samples can be taken from a subject and then immediately analyzed. Alternatively or in addition to, samples can be taken from a subject and analyzed at a later time. While awaiting analysis, samples can undergo various processing steps (e.g., filtration). While awaiting analysis, samples can be frozen. A frozen sample can be thawed prior to analysis. A sample can be analyzed about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more after collection from a subject.

In some embodiments, samples can be taken from a mammal, such as a dog, a cat, a horse, a monkey, an ape, a pig, a cow, a horse, a sheep, a goat. In some embodiments, samples can be taken from a human.

In some embodiments, a sample can be taken from a subject to determine breast cancer diagnosis. In some embodiments, a sample can be taken from a subject to confirm breast cancer diagnosis. In some embodiments, a sample can be taken from a subject to monitor breast cancer progression. In some embodiments, a sample can be taken from a subject to determine or confirm breast cancer remission.

### Biomarker Analysis

A biomarker is a biological molecule that is a sign of a normal or abnormal process, or of a condition or disease (e.g., breast cancer). A biomarker can be an DNA molecule, an RNA molecule (e.g., an RNA transcript suitable for RNA-Seq analysis), or a protein.

A biomarker can be identified as present in a sample. Alternatively, a biomarker can be identified as absent in sample. If a biomarker is present, it can be identified as being upregulated in a sample as compared to a control. Alternatively, if a biomarker is present, it can be identified as being downregulated in a sample as compared to a control. Alternatively, if a biomarker is present, it can be identified as being present in approximately the same level as a control.

A biomarker can be identified as being upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

A biomarker can be identified as being upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A biomarker can be identified as being upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

A biomarker can be identified as being downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

A biomarker can be identified as being downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A biomarker can be identified as being downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

Provided herein are antigens that are not expressed on leukocytes but which have a high and homogenous expression on the plasma membrane of breast cancer CTCs and which can be used as biomarkers for the reliable detection and characterization of CTCs. In some embodiments, the biomarkers used for detection of CTC include but are not limited to CD55, LSR, MARCKSL1, GPC1, SLC9A3R1, CXADR, SHROOM3, MUC1, PPP1R16A, ATP1B1, RHOD, SYTL2, or a combination thereof.

CD55 is the gene that encodes for complement decay-accelerating factor (also known as decay-accelerating factor, or DAF). CD55 regulates the complement system on the cell surface, and is a glycoprotein that is broadly distributed among hematopoietic and non-hematopoietic cells.

Lipolysis-stimulated lipoprotein receptor, LSR, is predicted to be involved in several processes, including establishment of skin barrier; protein localization to tricellular tight junction; and tricellular tight junction assembly.

MARCKSL1, also known as MARCKS like 1, is a member of the myristolyated alanine-rich C-kinase substrate (MARCKS) protein family. MARCKS proteins play a role in cytoskeletal regulation, protein kinase C signaling and calmodulin signaling, and MARCKSL1 affects the formation of adherens junctions.

Glypican 1, GPC1, is a cell surface heparan sulfate proteoglycan which is predicted to play a role in control of cell division and growth regulation.

SLC9A3R1, also known as NHERF family PDZ scaffold protein 1, is a sodium/hydrogen exchanger regulatory cofactor. SLC9A3R1 interacts with and regulates various proteins including the cystic fibrosis transmembrane conductance regulator and G-protein coupled receptors such as the beta2-adrenergic receptor and the parathyroid hormone 1 receptor. S LC9A3R1 also interacts with proteins that function as linkers between integral membrane and cytoskeletal proteins.

CXADR, also known as CXADR Ig-like cell adhesion molecule, is a type I membrane receptor for group B coxsackieviruses and subgroup C adenoviruses.

SHROOM3, also known as shroom family member 3, is a PDZ-domain-containing protein that belongs to a family of Shroom-related proteins. This protein may be involved in regulating cell shape in certain tissues.

Mucin short variant S1, also called MUC1, polymorphic epithelial mucin (PEM) or epithelial membrane antigen (EMA), is a glycoprotein that protects the body from infections.

Protein phosphatase 1 regulatory subunit 16A, PPP1R16A, is a part of a myosin light chain kinase and phosphatase (MLCP) complex which controls the phosphorylation states of regulatory myosin light chains, crucial for muscle and intracellular movement.

ATPase Na⁺/K⁺ transporting subunit beta 1, ATP1B1, is part of an integral membrane protein responsible for establishing and maintaining the electrochemical gradients of Na and K ions across the plasma membrane. These gradients are essential for osmoregulation, for sodium-coupled transport of a variety of organic and inorganic molecules, and for electrical excitability of nerve and muscle. This enzyme is composed of two subunits, a large catalytic subunit (alpha) and a smaller glycoprotein subunit (beta). The beta subunit regulates, through assembly of alpha/beta heterodimers, the number of sodium pumps transported to the plasma membrane.

Ras homolog gene family, member D, RHOD, is a GTPase that binds GTP and is involved in endosome dynamics and reorganization of the actin cytoskeleton, and it may coordinate membrane transport with the function of the cytoskeleton.

Synaptotagmin like 2, SYTL2, belongs to a C2 domain-containing protein family. The SLP homology domain (SHD) of this protein has been shown to specifically bind the GTP-bound form of Ras-related protein Rab-27A (RAB27A). This protein plays a role in RAB27A-dependent vesicle trafficking and controls melanosome distribution in the cell periphery.

The presence, absence, and/or differential level of at least one biomarker in a set of biomarkers in a sample can be indicative of breast cancer. At least one biomarker in a set of biomarkers can be present in a sample as compared to absent in a healthy control or reference sample to indicate the presence of breast cancer. Alternatively or in addition to, at least one biomarker in a set of biomarkers can be absent in a sample as compared to present in a healthy control or reference sample to indicate the presence of breast cancer. Alternatively or in addition to, at least one biomarker in a set of biomarkers can be upregulated in a sample as compared to a healthy control or reference sample to indicate the presence of breast cancer. Alternatively or in addition to, at least one biomarker in a set of biomarkers can be downregulated in a sample as compared to a healthy control or reference sample to indicate the presence of breast cancer. Alternatively or in addition to, at least one biomarker in a set of biomarkers can have the same or similar differential level in a sample as compared to a reference sample to indicate the presence of breast cancer. In some embodiments, the information of one biomarker can be used to diagnose breast cancer. Alternatively, in some embodiments, the information of multiple biomarkers can be combined to diagnose breast cancer.

A set of biomarkers can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 85, at least 90, at least 95, at least 100, at least 150, at least 200, or more biomarkers. In some embodiments, the set of biomarkers can comprise one biomarker. In some embodiments, the set of biomarkers can comprise two biomarkers. In some embodiments, the set of biomarkers can comprise three biomarkers. In some embodiments, the set of biomarkers can comprise four biomarkers. In some embodiments, the set of biomarkers can comprise five biomarkers. In some embodiments, the set of biomarkers can comprise six biomarkers. In some embodiments, the set of biomarkers can comprise seven biomarkers. In some embodiments, the set of biomarkers can comprise eight biomarkers. In some embodiments, the set of biomarkers can comprise nine biomarkers. In some embodiments, the set of biomarkers can comprise ten biomarkers. In some embodiments, the set of biomarkers can comprise eleven biomarkers. In some embodiments, the set of biomarkers can comprise twelve biomarkers.

In some embodiments, the set of biomarkers comprises CD55. In some embodiments, the set of biomarkers comprises LSR. In some embodiments, the set of biomarkers comprises MARCKSL1. In some embodiments, the set of biomarkers comprises GPC1. In some embodiments, the set of biomarkers comprises SLC9A3R1. In some embodiments, the set of biomarkers comprises CXADR. In some embodiments, the set of biomarkers comprises SHROOM3. In some embodiments, the set of biomarkers comprises MUC1. In some embodiments, the set of biomarkers comprises PPP1R16A. In some embodiments, the set of biomarkers comprises ATP1B1. In some embodiments, the set of biomarkers comprises RHOD. In some embodiments, the set of biomarkers comprises SYTL2. In some embodiments, the set of biomarkers comprises CD55 and LSR. In some embodiments, the set of biomarkers comprises CD55 and MARCKSL1. In some embodiments, the set of biomarkers comprises CD55 and GPC1. In some embodiments, the set of biomarkers comprises CD55 and SLC9A3R1. In some embodiments, the set of biomarkers comprises CD55 and CXADR. In some embodiments, the set of biomarkers comprises CD55 and SHROOM3. In some embodiments, the set of biomarkers comprises CD55 and MUC1. In some embodiments, the set of biomarkers comprises CD55 and PPP1R16A. In some embodiments, the set of biomarkers comprises CD55 and ATP1B1. In some embodiments, the set of biomarkers comprises CD55 and RHOD. In some embodiments, the set of biomarkers comprises CD55 and SYTL2. In some embodiments, the set of biomarkers comprises LSR and MARCKSL1. In some embodiments, the set of biomarkers comprises LSR and GPC1. In some embodiments, the set of biomarkers comprises LSR and SLC9A3R1. In some embodiments, the set of biomarkers comprises LSR and CXADR. In some embodiments, the set of biomarkers comprises LSR and SHROOM3. In some embodiments, the set of biomarkers comprises LSR and MUC1. In some embodiments, the set of biomarkers comprises LSR and PPP1R16A. In some embodiments, the set of biomarkers comprises LSR and ATP1B1. In some embodiments, the set of biomarkers comprises LSR and RHOD. In some embodiments, the set of biomarkers comprises LSR and SYTL2. In some embodiments, the set of biomarkers comprises MARCKSL1 and GPC1. In some embodiments, the set of biomarkers comprises MARCKSL1 and SLC9A3R1. In some embodiments, the set of biomarkers comprises MARCKSL1 and CXADR. In some embodiments, the set of biomarkers comprises MARCKSL1 and SHROOM3. In some embodiments, the set of biomarkers comprises MARCKSL1 and MUC1. In some embodiments, the set of biomarkers comprises MARCKSL1 and PPP1R16A. In some embodiments, the set of biomarkers comprises MARCKSL1 and ATP1B1. In some embodiments, the set of biomarkers comprises MARCKSL1 and RHOD. In some embodiments, the set of biomarkers comprises MARCKSL1 and SYTL2. In some embodiments, the set of biomarkers comprises GPC1 and SLC9A3R1. In some embodiments, the set of biomarkers comprises GPC1 and CXADR. In some embodiments, the set of biomarkers comprises GPC1 and SHROOM3. In some embodiments, the set of biomarkers comprises GPC1 and MUC1. In some embodiments, the set of biomarkers comprises GPC1 and PPP1R16A. In some embodiments, the set of biomarkers comprises GPC1 and ATP1B1. In some embodiments, the set of biomarkers comprises GPC1 and RHOD. In some embodiments, the set of biomarkers comprises GPC1 and SYTL2. In some embodiments, the set of biomarkers comprises SLC9A3R1 and CXADR. In some embodiments, the set of biomarkers comprises SLC9A3R1 and SHROOM3. In some embodiments, the set of biomarkers comprises SLC9A3R1 and MUC1. In some embodiments, the set of biomarkers comprises SLC9A3R1 and PPP1R16A. In some embodiments, the set of biomarkers comprises SLC9A3R1 and ATP1B1. In some embodiments, the set of biomarkers comprises SLC9A3R1 and RHOD. In some embodiments, the set of biomarkers comprises SLC9A3R1 and SYTL2. In some embodiments, the set of biomarkers comprises CXADR and SHROOM3. In some embodiments, the set of biomarkers comprises CXADR and MUC1. In some embodiments, the set of biomarkers comprises CXADR and PPP1R16A. In some embodiments, the set of biomarkers comprises CXADR and ATP1B1. In some embodiments, the set of biomarkers comprises CXADR and RHOD. In some embodiments, the set of biomarkers comprises CXADR and SYTL2. In some embodiments, the set of biomarkers comprises SHROOM3 and MUC1. In some embodiments, the set of biomarkers comprises SHROOM3 and PPP1R16A. In some embodiments, the set of biomarkers comprises SHROOM3 and ATP1B1. In some embodiments, the set of biomarkers comprises SHROOM3 and RHOD. In some embodiments, the set of biomarkers comprises SHROOM3 and SYTL2. In some embodiments, the set of biomarkers comprises MUC1 and PPP1R16A. In some embodiments, the set of biomarkers comprises MUC1 and ATP1B1. In some embodiments, the set of biomarkers comprises MUC1 and RHOD. In some embodiments, the set of biomarkers comprises MUC1 and SYTL2. In some embodiments, the set of biomarkers comprises PPP1R16A and ATP1B1. In some embodiments, the set of biomarkers comprises PPP1R16A and RHOD. In some embodiments, the set of biomarkers comprises PPP1R16A and SYTL2. In some embodiments, the set of biomarkers comprises RHOD and SYTL2. In some embodiments, the set of biomarkers comprises MARCKSL1, SLC9A3R1, and ATP18. In some embodiments, the set of biomarkers comprises MARCKSL1, SLC9A3R1, and RHOD. In some embodiments, the set of biomarkers comprises MARCKSL1, SLC9A3R1, ATP18, and RHOD. In some embodiments, the set of biomarkers comprises MARCKSL1, SLC9A3R1, ATP18, RHOD, and LSR. In some embodiments, the set of biomarkers comprises CD55, GPC1, CXADR, SHROOM3, MUC1, PPP1R16A, and SYTL2. In some embodiments, the set of biomarkers comprises CD55, LSR, MARCKSL1, GPC1, SLC9A3R1, CXADR, SHROOM3, MUC1, PPP1R16A, ATP1B1, RHOD, and SYTL2. In some embodiments, the set of biomarkers comprises CD55, LSR, MARCKSL1, GPC1, SLC9A3R1, CXADR, SHROOM3, MUC1, PPP1R16A, ATP1B1, RHOD, SYTL2, or any combination thereof.

In some embodiments, CD55 is differentially regulated in a sample as compared to a healthy control or reference sample to indicate the presence of breast cancer. In some embodiments, CD55 is upregulated in a sample as compared to a healthy control or reference sample to indicate the presence of breast cancer. In some embodiments, CD55 is downregulated in a sample as compared to a healthy control or reference sample to indicate the presence of breast cancer.

In some embodiments, CD55 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, CD55 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, CD55 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, CD55 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, CD55 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, CD55 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, LSR is differentially regulated in a sample as compared to a healthy control or reference sample to indicate the presence of breast cancer. In some embodiments, LSR is upregulated in a sample as compared to a healthy control or reference sample to indicate the presence of breast cancer. In some embodiments, LSR is downregulated in a sample as compared to a healthy control or reference sample to indicate the presence of breast cancer.

In some embodiments, LSR is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, LSR is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, LSR is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, LSR is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, LSR is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, LSR is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, MARCKSL1 is differentially regulated in a sample as compared to a healthy control or reference sample to indicate the presence of breast cancer. In some embodiments, MARCKSL1 is upregulated in a sample as compared to a healthy control or reference sample to indicate the presence of breast cancer. In some embodiments, MARCKSL1 is downregulated in a sample as compared to a healthy control or reference sample to indicate the presence of breast cancer.

In some embodiments, MARCKSL1 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, MARCKSL1 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, MARCKSL1 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, MARCKSL1 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, MARCKSL1 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, MARCKSL1 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, GPC1 is differentially regulated in a sample as compared to a healthy control or reference sample to indicate the presence of breast cancer. In some embodiments, GPC1 is upregulated in a sample as compared to a healthy control or reference sample to indicate the presence of breast cancer. In some embodiments, GPC1 is downregulated in a sample as compared to a healthy control or reference sample to indicate the presence of breast cancer.

In some embodiments, GPC1 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, GPC1 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, GPC1 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, GPC1 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, GPC1 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, GPC1 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, SLC9A3R1 is differentially regulated in a sample as compared to a healthy control or reference sample to indicate the presence of breast cancer. In some embodiments, SLC9A3R1 is upregulated in a sample as compared to a healthy control or reference sample to indicate the presence of breast cancer. In some embodiments, SLC9A3R1 is downregulated in a sample as compared to a healthy control or reference sample to indicate the presence of breast cancer.

In some embodiments, SLC9A3R1 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, SLC9A3R1 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, SLC9A3R1 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, SLC9A3R1 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, SLC9A3R1 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, SLC9A3R1 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, CXADR is differentially regulated in a sample as compared to a healthy control or reference sample to indicate the presence of breast cancer. In some embodiments, CXADR is upregulated in a sample as compared to a healthy control or reference sample to indicate the presence of breast cancer. In some embodiments, CXADR is downregulated in a sample as compared to a healthy control or reference sample to indicate the presence of breast cancer.

In some embodiments, CXADR is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, CXADR is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, CXADR is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, CXADR is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, CXADR is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, CXADR is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, SHROOM3 is differentially regulated in a sample as compared to a healthy control or reference sample to indicate the presence of breast cancer. In some embodiments, SHROOM3 is upregulated in a sample as compared to a healthy control or reference sample to indicate the presence of breast cancer. In some embodiments, SHROOM3 is downregulated in a sample as compared to a healthy control or reference sample to indicate the presence of breast cancer.

In some embodiments, SHROOM3 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, SHROOM3 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, SHROOM3 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, SHROOM3 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, SHROOM3 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, SHROOM3 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, MUC1 is differentially regulated in a sample as compared to a healthy control or reference sample to indicate the presence of breast cancer. In some embodiments, MUC1 is upregulated in a sample as compared to a healthy control or reference sample to indicate the presence of breast cancer. In some embodiments, MUC1 is downregulated in a sample as compared to a healthy control or reference sample to indicate the presence of breast cancer.

In some embodiments, MUC1 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, MUC1 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, MUC1 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, MUC1 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, MUC1 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, MUC1 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, PPP1R16A is differentially regulated in a sample as compared to a healthy control or reference sample to indicate the presence of breast cancer. In some embodiments, PPP1R16A is upregulated in a sample as compared to a healthy control or reference sample to indicate the presence of breast cancer. In some embodiments, PPP1R16A is downregulated in a sample as compared to a healthy control or reference sample to indicate the presence of breast cancer.

In some embodiments, PPP1R16A is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, PPP1R16A is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, PPP1R16A is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, PPP1R16A is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, PPP1R16A is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, PPP1R16A is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, ATP1B1 is differentially regulated in a sample as compared to a healthy control or reference sample to indicate the presence of breast cancer. In some embodiments, ATP1B1 is upregulated in a sample as compared to a healthy control or reference sample to indicate the presence of breast cancer. In some embodiments, ATP1B1 is downregulated in a sample as compared to a healthy control or reference sample to indicate the presence of breast cancer.

In some embodiments, ATP1B1 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, ATP1B1 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, ATP1B1 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, ATP1B1 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, ATP1B1 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, ATP1B1 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, RHOD is differentially regulated in a sample as compared to a healthy control or reference sample to indicate the presence of breast cancer. In some embodiments, RHOD is upregulated in a sample as compared to a healthy control or reference sample to indicate the presence of breast cancer. In some embodiments, RHOD is downregulated in a sample as compared to a healthy control or reference sample to indicate the presence of breast cancer.

In some embodiments, RHOD is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, RHOD is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, RHOD is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, RHOD is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, RHOD is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, RHOD is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, SYTL2 is differentially regulated in a sample as compared to a healthy control or reference sample to indicate the presence of breast cancer. In some embodiments, SYTL2 is upregulated in a sample as compared to a healthy control or reference sample to indicate the presence of breast cancer. In some embodiments, SYTL2 is downregulated in a sample as compared to a healthy control or reference sample to indicate the presence of breast cancer.

In some embodiments, SYTL2 is upregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being upregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, SYTL2 is upregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, SYTL2 is upregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, SYTL2 is downregulated by at least about 5%, at least about 10%, at least about 15%, at least about 20%, at least about 30%, at least about 40%, at least about 50%, at least about 60%, at least about 70%, at least about 80%, at least about 90%, at least about 100%, at least about 500%, at least about 1,000%, at least about 5,000%, at least about 10,000%, or more. A biomarker can be identified as being downregulated by at most about 10,000%, at most about 5,000%, at most about 1,000%, at most about 500%, at most about 100%, at most about 90%, at most about 80%, at most about 70%, at most about 60%, at most about 50%, at most about 40%, at most about 30%, at most about 20%, at most about 15%, at most about 10%, at most about 5% or less.

In some embodiments, SYTL2 is downregulated by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. In some embodiments, SYTL2 is downregulated by at most or up to about 10,000-fold, at most or up to about 5,000-fold, at most up to or about 1,000-fold, at most up to or about 500-fold, at most up to or about 100-fold, at most up to or about 90-fold, at most up to or about 80-fold, at most up to or about 70-fold, at most up to or about 60-fold, at most up to or about 50-fold, at most up to or about 40-fold, at most up to or about 30-fold, at most up to or about 20-fold, at most up to or about 10-fold, at most up to or about 9-fold, at most up to or about 8-fold, at most up to or about 7-fold, at most up to or about 6-fold, at most up to or about 5-fold, at most up to or about 4-fold, at most up to or about 3-fold, at most up to or about 2-fold, at most up to or about 1-fold, at most up to or about 0.9-fold, at most up to or about 0.8-fold, at most up to or about 0.7-fold, at most up to or about 0.6-fold, at most up to or about 0.5-fold, at most up to or about 0.4-fold, at most up to or about 0.3-fold, at most up to or about 0.2-fold, or at most up to or about 0.1-fold.

In some embodiments, housekeeping genes or proteins, such as CD45, are used as a control. Alternatively or in addition to, epithelial-specific markers such as epithelial cellular adhesion molecules (EpCAM) and/or cytokeratin (CK) can be used as a control.

### Kits

Provided herein, in some aspects, are kits comprising the one or more compositions disclosed herein. In some embodiments, the kits comprise one or more biomarkers or controls described herein. In some embodiments, the kits comprise one or more antibodies to the biomarkers or controls described herein. In some embodiments, the kits further comprise materials for the isolation of DNA and RNA, materials for sequencing preparation, materials for amplification preparation (e.g., PCR), and materials for immunofluorescence staining. In some embodiments, the kits comprise materials for isolating and filtering samples (e.g., blood samples). In some embodiments, the kits further comprise a cell, a plurality of cells, or a cell line such as a breast cancer cell line (e.g., SKBR3 or MCF7). The exact nature of the components configured in the inventive kit depends on its intended purpose.

Instructions for use may be included in the kit. “Instructions for use” typically include a tangible expression describing the technique to be employed in using the components of the kit to effect a desired outcome, e.g., determining the presence, absence, or differential level of a biomarker in a sample as described herein. Optionally, the kit also contains other useful components, such as, diluents, buffers, pharmaceutically acceptable carriers, syringes, tubes, vials, cuvettes, filters, pipetting or measuring tools, or other useful paraphernalia as will be readily recognized by those of skill in the art.

The materials or components assembled in the kit can be provided to the user stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a plastic vial or tube used to contain suitable quantities of the antibodies and/or cells. The packaging material generally has an external label which indicates the contents and/or purpose of the kit and/or its components.

### Computer Systems

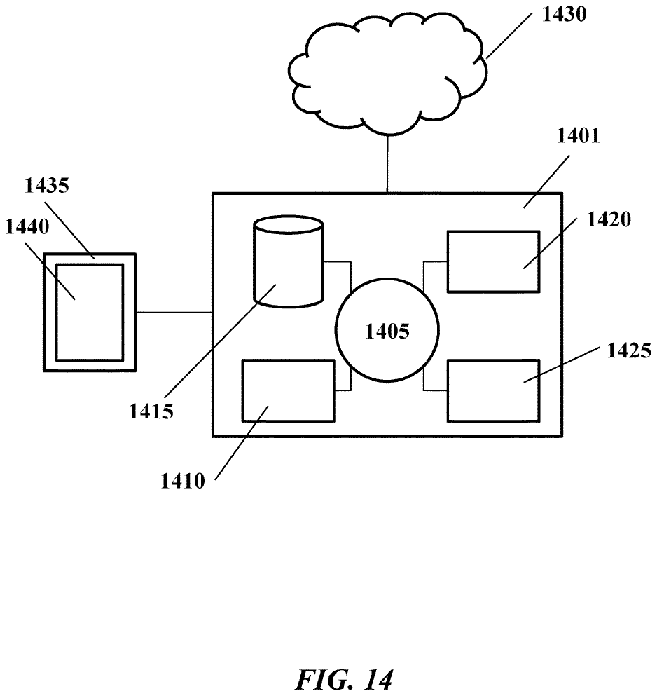

The present disclosure provides computer systems that are programmed to implement methods of the disclosure. FIG. 14 shows a computer system 1401 that is programmed or otherwise configured to analyze results of differential levels of one or more biomarkers. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, results on differential levels of one or more biomarkers. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, analyze results on differential levels of one or more biomarkers.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Identification of Biomarkers Using in Silico Bioinformatics

Bioinformatics was used to identify common antigens that are overexpressed in three different sources: 1) primary breast cancer tumor cells 2), breast cancer CTCs, and 3) the most common breast cancer cell lines (as these are easily accessible and essential elements in cancer research). The inclusion of common breast cancer cell lines also facilitates the utilization of breast cancer cell lines that expresses antigens of interest for further in vitro experiments.

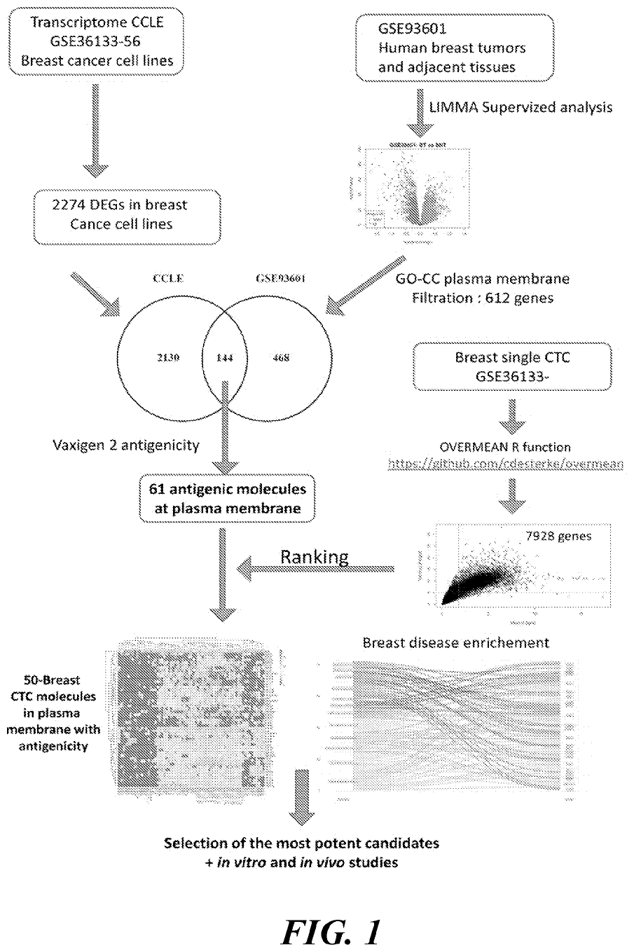

To initiate this study, a specific bioinformatics workflow was established for breast cancer transcriptome analysis (FIG. 1). First, overexpressed breast primary tumor markers were explored for their heterogeneity of expression in breast cancer cell lines. This led to the identification of 2274 genes. In parallel, a transcriptome-supervised analysis was used to identify 612 genes that are both upregulated in breast primary tumors in comparison to breast adjacent tissue samples and known to be present on the plasma membrane by querying the Gene Ontology Cell Compartment database. Second, in following downstream analysis, the plasma membrane molecules signature was merged with the first gene profile found across breast cancer cell lines to find overlapping molecules. This process led to the identification of 144 commonly upregulated genes. Third, breast cancer's differentially expressed gene (DEG) profile was restricted to antigenic molecules evaluating the antigenicity of their sequence with the Vaxijen score. This bioinformatic toolkit maintained 61 out of 144 genes that were initially identified.

Finally, the expression of common breast cancer antigenic markers was compared to breast cancer CTCs. A complete single-cell transcriptome from a unique CTC dataset (GSE109761) was analyzed with “overmean” R-function. This analysis revealed that breast cancer CTCs expressed 7928 DEGs. Merging single-cell transcriptome analysis of CTCs with 61 overexpressed antigenic molecules in primary breast tumors demonstrated 50 of them were expressed in the majority of CTCs (FIG. 1). After functional enrichment of these CTC antigenic markers on the DisGeNet disease database, some of the genes were found to be already known to be associated with breast neoplasm disorders, especially invasive ones. These results suggest that antigenic plasma membrane markers of breast cancer CTCs reflect an invasive feature of breast neoplasm disorders. This bioinformatic workflow narrowed the number of potential markers down to 50 candidates.

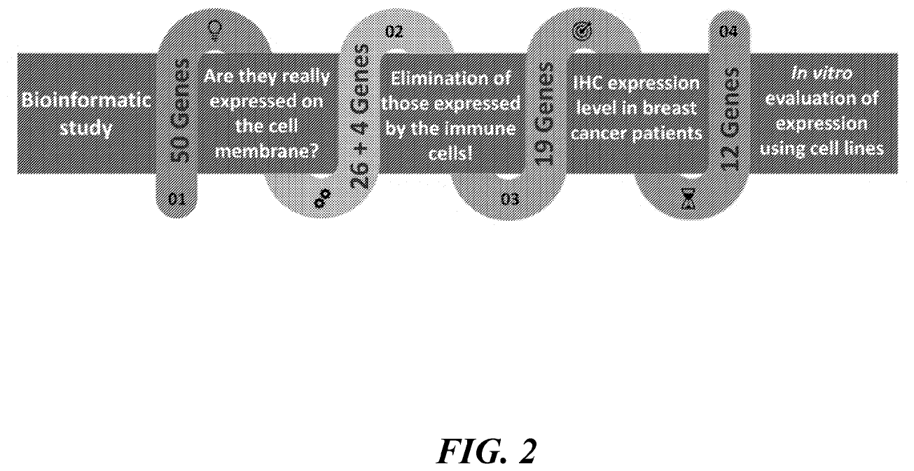

A series of in silico filtrations was performed to preserve only the most significant identified targets (FIG. 2). First, the selected 50 genes were verified as truly being expressed on the cell cytoplasmic membrane. To do such, two databases (Protein Atlas and UniProt) were used. Data showed that 26 out of the 50 genes had membrane expression according to both databases, and 4 out of the 50 genes had membrane expression according to only one of the databases. Those 30 genes were selected as potential candidates.

In the next step, blood samples were filtered. After blood filtration, CTCs were retained on the isolation support along with a small fraction of larger leukocytes. This allowed for elimination of the genes that were expressed by immune cells as determined by the two databases in order to have the maximum CTC specificity, a process that led to the preservation of 19 target genes.

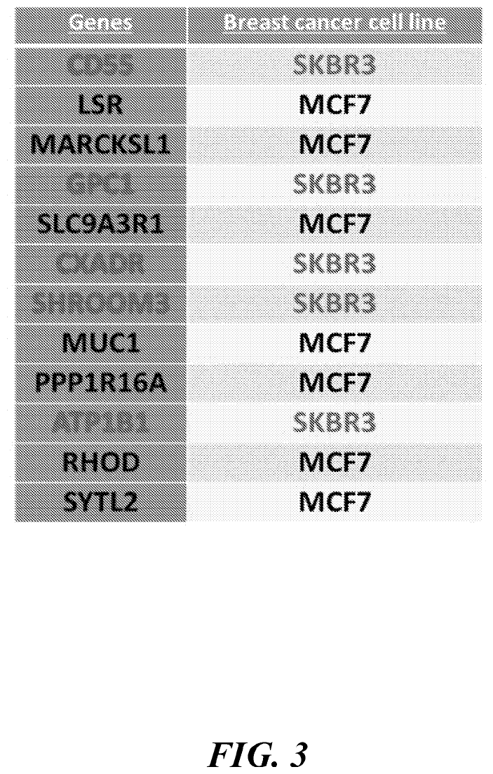

Only 12 genes (CD55, LSR, MARCKSL1, GPC1, SLC9A3R1, CXADR, SHROOM3, MUC1, PPP1R16A, ATP1B1, RHOD, and SYTL2) were kept because, according to the databases, they had an intermediate or high expression level (performed by immunohistochemistry, (IHC)) in several primary tumor samples of breast cancer patients. RNA sequencing data of different breast cancer cell lines (Protein Atlas) and the inclusion of breast cancer cell lines in this study enabled inspection of which cell lines expressed the 12 target genes. MCF7 and SKBR3 breast cancer cell lines were found to express all of the genes of interest (FIG. 3).

### Example 2: In Vivo Identification of Breast Cancer Using Biomarkers

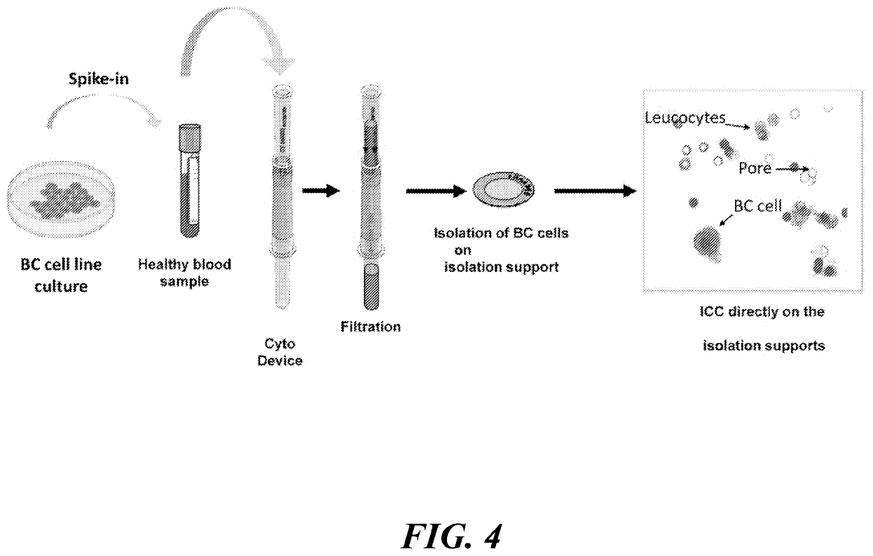

Upon termination of in silico studies, the in vitro validation began. 5000 breast cancer cell lines were spiked into 3 ml of healthy blood samples and those cells were captured on isolation supports. A high number of cells was used to ensure the target genes were correctly expressed at the protein level. Once cells were captured, Immunocytochemistry (ICC) methods were used to label leucocytes with anti-C45/DAB and breast cancer cells with anti-CK18/RED (FIG. 4). SKBR3 cells were also labelled using an anti-CK 8 antibody.

A series of ICC stainings were performed using breast cancer cell lines and enabled the expression level of all 12 genes of interests to be confirmed at the protein level. Special attention was paid to the number of positive cells, the intensity of the staining, membrane expression, and the specificity of each targeted protein (including absence of expression in leucocytes).

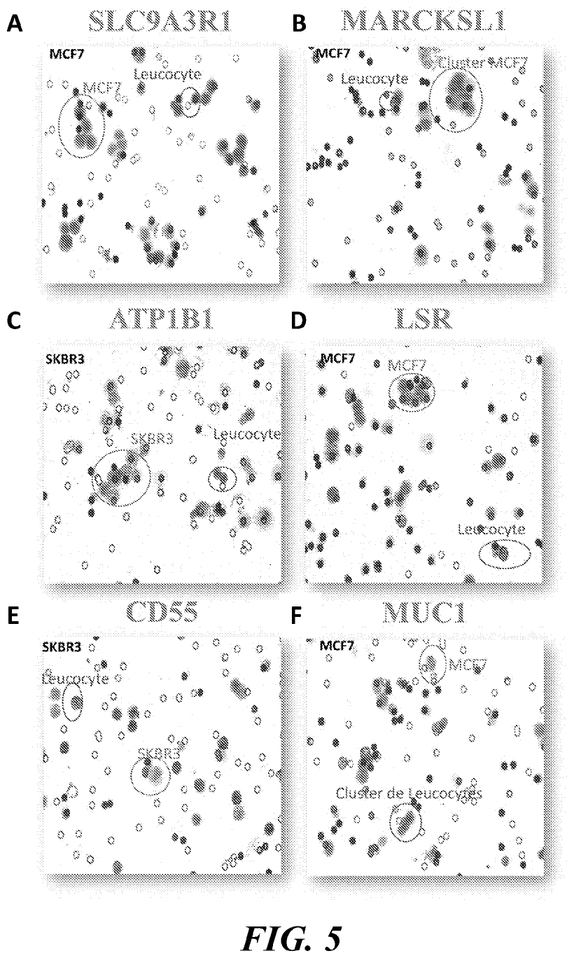

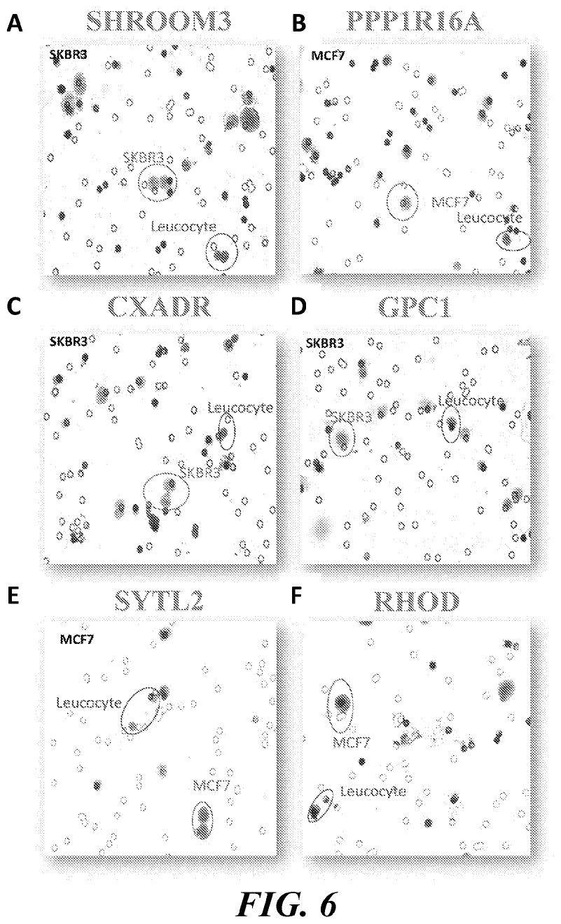

FIG. 5 and FIG. 6 show representative ICC staining targeting all 12 proteins in the related breast cancer cell lines.

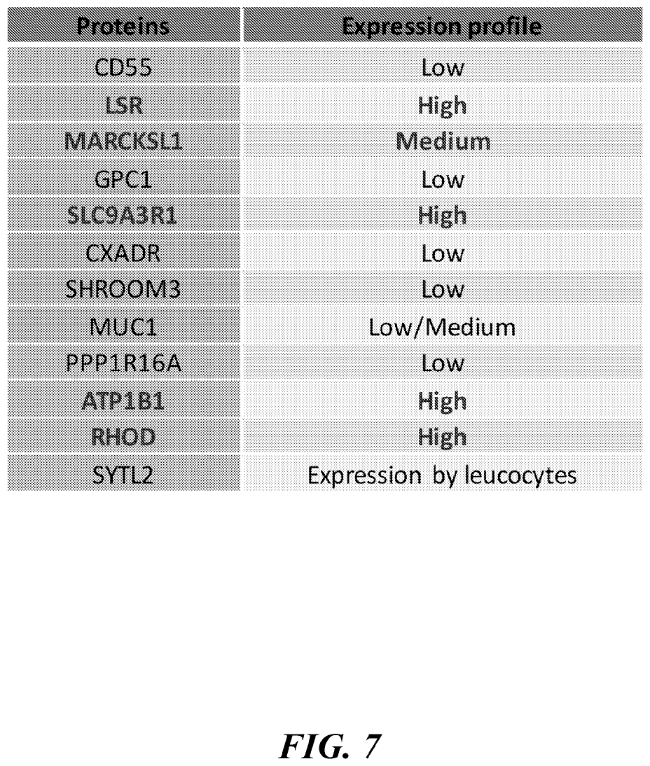

These experiments revealed that 5 out of the 12 proteins had a higher expression level, while the rest had a lower expression level. FIG. 7 summarize the expression profile of each protein.

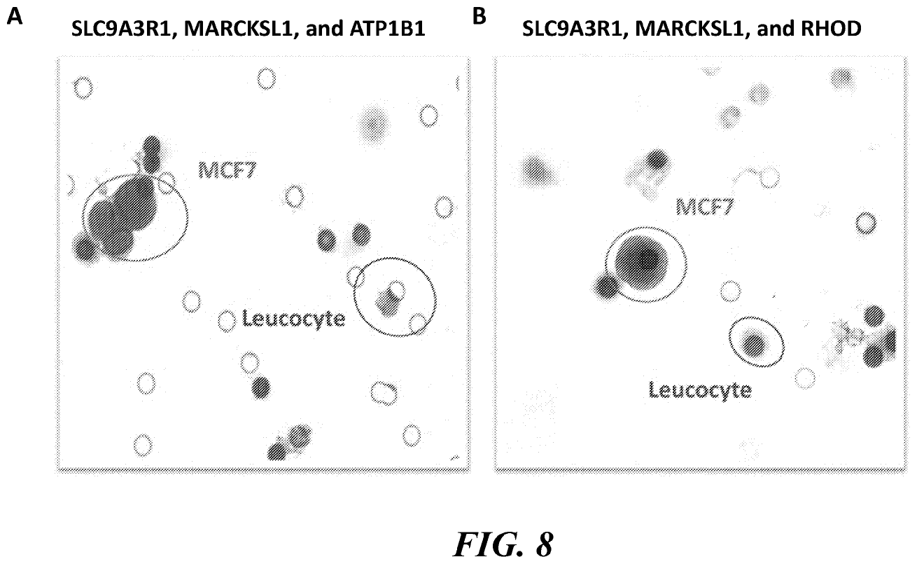

The same experimental procedure was repeated but this time 20 breast cancer cells were spiked into 3 ml of healthy blood samples in order to more closely mimic a patient's blood condition (CTC concentrations are very low, approximately 1 to 50 CTCs per 3 ml of blood). The five proteins with higher expression levels were selected and it was demonstrated that while MARCKSL1, SLC9A3R1, ATP1B1, and RHOD are highly positive in the majority of breast cancer cell lines, many cells remained unstained while using anti-LSR antibodies. Two combinations of these markers, either MARCKSL1, SLC9A3R1, and ATP1B1 (Cocktail 1) or MARCKSL1, SLC9A3R1, and RHOD (Cocktail 2) were used together in a trio cocktail to target the highest number of cells with the uppermost intensity of staining (FIG. 8). The mixture of antibodies with the same color led to the most successful ICC staining, validating these 4 markers for further in vivo confirmations.

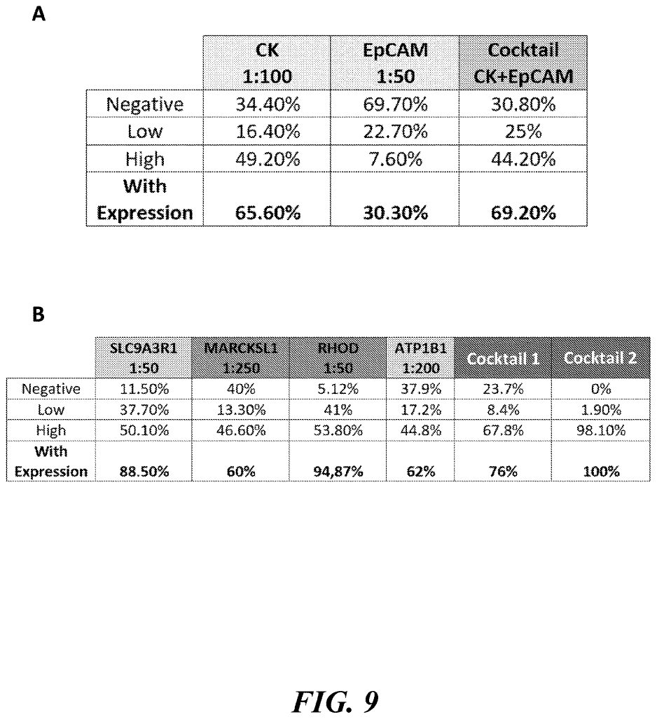

FIG. 9 compared the efficacy of each marker separately and in combinations, along with epithelial controls.

### Example 3: Identification of Breast Cancer Using Biomarker Cocktail in Humans

Eleven lymph node or distant metastatic breast cancer patients were recruited to investigate the clinical validity of the novel biomarkers.

K2-EDTA tubes of 9 ml (18 ml of blood in total) were drawn per patient. The blood was processed locally in a devoted lab and 5 isolation supports (5 times 3 ml) were prepared for each patient using ScreenCell Cyto kits (55 isolation support in total).

One isolation support sample stained the captured cells with EpbCAM+CK (conventional markers) in addition to CD45 staining, which was performed for all samples. Three isolation supports were dedicated to assessing the expression of 4 new biomarkers individually (MARCKSL1, SLC9A3R1, RHOD, and ATP1B1). The last isolation support was used to stain the cells with a cocktail of the best biomarkers simultaneously.

The analysis of all 55 filters by a certified pathologist revealed that a potential CTC (denoted by abnormally large cells) was present in every one of the samples, confirming that all samples could be used for further ICC analysis.

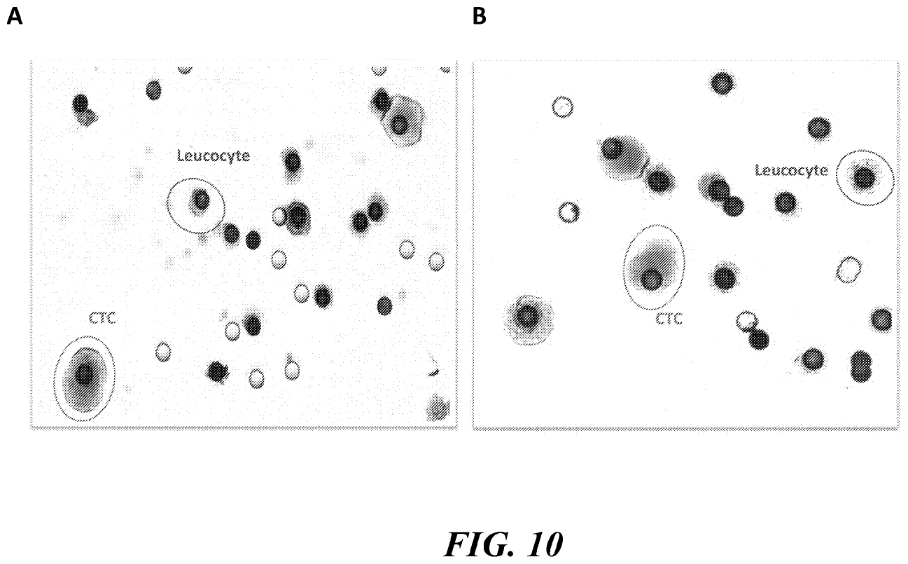

As anticipated, ICC staining results on patient samples demonstrated a diverse expression of the EpCAM+CK conventional markers, proving the vulnerability of these markers in CTC detection. In this setting, while some cells expressed these markers others remained slightly stained or even negative (FIG. 10).

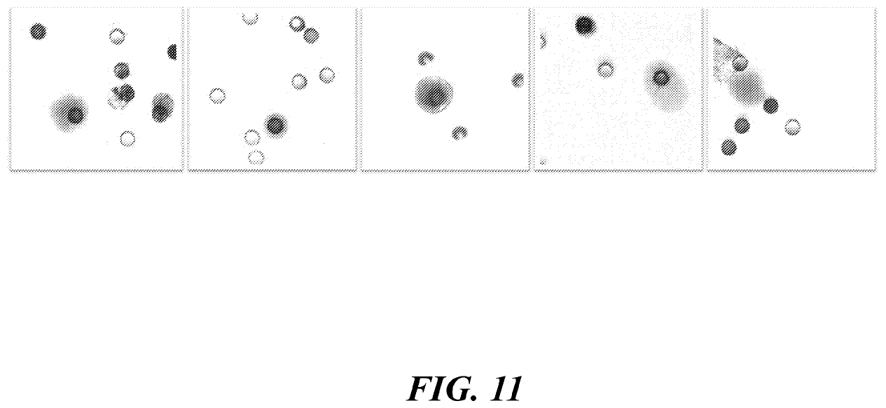

Since the number of potential biomarkers (4 biomarkers) was more than the number of assigned isolation supports (3 isolation supports), the samples were labeled with individual biomarkers. Interestingly, it was observed that, unlike its strong expression in cell lines, ATP1A1 was completely negative when targeted in 5 different breast cancer patients (FIG. 11). The other 3 markers (MARCKSL1, SLC9A3R1, and RHOD) showed positive but heterogenous expression when used separately.

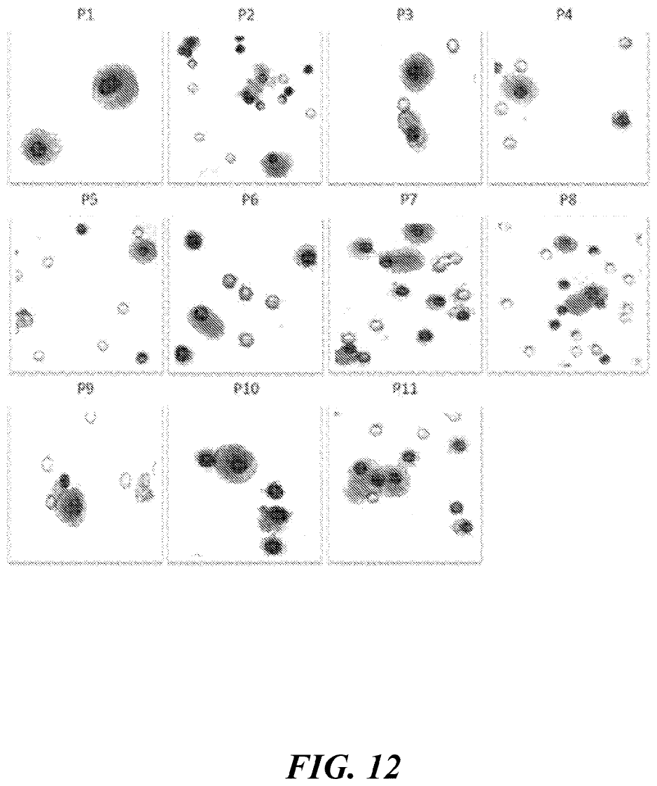

Therefore, the three markers, MARCKSL1, SLC9A3R1, and RHOD, were combined into a cocktail to maximize their labeling capacity. Interestingly, results demonstrated the most effective, boosted, and more homogenous immunostaining of CTCs while applying this antibody cocktail. In this setting, almost 100% of the CTCs in all 11 patients were labeled leading to the discovery of a set of reliable biomarkers to distinguish breast cancer CTCs from the remaining leucocytes (FIG. 12).

### Example 4: Identification of Breast Cancer Using Biomarker Cocktail in Humans

Forty lymph node or distant metastatic breast cancer patients and 20 healthy donors (from the French National Blood Service) were recruited to investigate the specificity of the novel biomarkers.

Up to 2 K2-EDTA tubes of 9 ml (between 9 ml and 18 ml of blood in total) were drawn per patient. The blood taking was processed locally in a devoted lab and 3 to 5 isolation supports (3 ml of blood per isolation support) were prepared for each patient using ScreenCell Cyto kits (202 isolation supports in total).

For the first 11 patients with 5 isolation supports, the first isolation support was used to stain the captured cells with EpCAM+CK (conventional markers) in addition to CD45 staining that was performed for all samples. Three isolation supports were dedicated to assessing the expression of 4 new markers individually (MARCKSL1, SLC9A3R1, RHOD, and ATP1B1). The last isolation support was used to stain the cells with a cocktail of the best markers simultaneously.

ICC staining results on patient samples demonstrated a diverse expression of the EpCAM+CK conventional markers, proving the limitation of these markers in CTC detection. In this setting, while some cells expressed these markers others remained slightly labelled or even negative.

Since the number of potential markers to test (4 biomarkers) was more than the assigned isolation supports (3 isolation supports), labeling of the samples began with individual biomarkers. It was observed that, unlike its strong expression in cell lines, ATP1A1 was completely negative when targeted in 5 different BC patients. The other 3 markers (MARCKSL1, SLC9A3R1, and RHOD) showed positive but heterogenous expression when used separately.

Therefore, those 3 markers (MARCKSL1, SLC9A3R1, and RHOD) were combined into a cocktail (Cocktail 2) to maximize their labeling capacity.

After the selection of the most efficient markers, 20 out of 40 patients were analyzed.

Primarily, the cytopathological analysis of these 20 patients by a certified pathologist revealed that potential CTCs (according to morphological criteria) were present in every one of the samples, reassuring that all samples could be used for further ICC analysis.

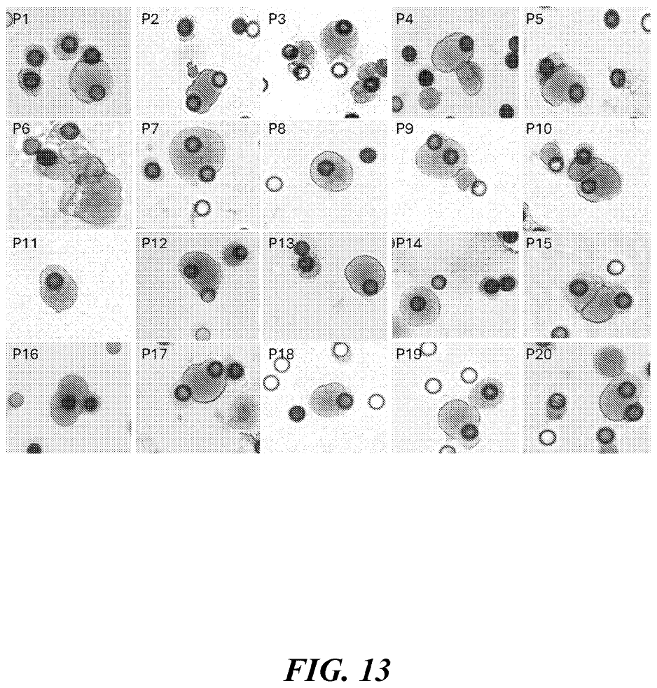

ICC labelling using the cocktail 2 markers demonstrated the most effective, strong, and more homogenous immunostaining of CTCs compared to CK+EpCAM markers. In this setting, the average number of CTCs after ICC staining using our markers was 27.65 CTCs/patient. More precisely, in the blood samples of these 20 patients, 553 atypical cells were found and among them, 549 cells (more than 99%) were positively stained using the cocktail markers (FIG. 13). The analysis of the remaining patients is still ongoing.

Concerning the CK+EpCAM markers in those 20 patients, a total number of 444 atypical cells were found based on morphological analysis, and the ICC staining validated 311 (70%) of the positive cells. The analysis of the rest of the patients is still ongoing.

These preliminary results demonstrate that the markers are significantly stronger than conventional markers to identify CTCs.

Additionally, the morphological analysis of 15 out of 20 healthy donors demonstrates a significantly lower number of atypical cells (1.53 on average) showing a significant difference between healthy donors and cancer patient blood.

### Example 5: In Vivo Identification of Breast Cancer Using Biomarker Cocktail

In this prophetic example, forty lymph node or distant metastatic breast cancer patients and 20 healthy donors (from the French National Blood Service) are recruited to investigate the specificity of the novel biomarkers.

Up to 2 K2-EDTA tubes of 9 ml (between 9 ml and 18 ml of blood in total) are drawn per patient. The blood taking is processed locally in a devoted lab and 3 to 5 isolation supports (3 ml of blood per isolation support) are prepared for each patient using ScreenCell Cyto kits (202 isolation supports in total).

For the first 11 patients with 5 isolation supports, the first isolation support is used to stain the captured cells with EpCAM+CK (conventional markers) in addition to CD45 staining that is performed for all samples. Three isolation supports are dedicated to assessing the expression of twelve new markers individually (CD55, LSR, MARCKSL1, GPC1, SLC9A3R1, CXADR, SHROOM3, MUC1, PPP1R16A, ATP1B1, RHOD, and SYTL2). The last isolation support is used to stain the cells with a cocktail of the 3 best markers simultaneously, wherein the cocktail is not Cocktail 1 or Cocktail 2. Subsequent experiments will further stain additional cells with cocktails of additional marker combinations. See Table 1.

TABLE 1
Biomarker Cocktails

| Biomarker | |
|---|---|
| Cocktail | Included Biomarkers |
| Cocktail 1 | MARCKSL1, SLC9A3R1, ATP1B1 |
| Cocktail 2 | MARCKSL1, SLC9A3R1, RHOD |
| Cocktail 3 | ATP1B1, RHOD, MARCKSL1 |
| Cocktail 4 | ATP1B1, RHOD, SLC9A3R1 |
| Cocktail 5 | CXADR, SHROOM3, MUC1, PPP1R16A |
| Cocktail 6 | MARCKSL1, SLC9A3R1, SHROOM3 |
| Cocktail 7 | MARCKSL1, SLC9A3R1, MUC1, SHROOM3 |
| Cocktail 8 | MARCKSL1, SLC9A3R1, RHOD, MUC1 |
| Cocktail 9 | MARCKSL1, SLC9A3R1, RHOD, GPC1 |
| Cocktail 10 | MARCKSL1, SLC9A3R1, RHOD, PPP1R16A |
| Cocktail 11 | MARCKSL1, SLC9A3R1, RHOD, ATP1B1 |
| Cocktail 12 | MARCKSL1, SLC9A3R1, RHOD, ATP1B1, LSR |
| Cocktail 13 | CD55, GPC1, CXADR, SHROOM3, MUC1, PPP1R16A |
| Cocktail 14 | CD55, GPC1, CXADR, SHROOM3, MUC1, PPP1R16A, |
| | SYTL2 |
| Cocktail 15 | CD55, LSR, MARCKSL1, GPC1, SLC9A3R1, CXADR, |
| | SHROOM3, MUC1, PPP1R16A, ATP1B1, RHOD, SYTL2 |

ICC staining results using EpCAM and/or CD on patient samples provide a control.

Since the number of potential markers to test (4 biomarkers) is more than the assigned isolation supports (3 isolation supports), labeling of the samples begins with individual biomarkers.

After the selection of the most efficient markers, 20 out of 40 patients were analyzed.

ICC labelling using the chosen cocktail markers demonstrate the most effective, strong, and more homogenous immunostaining of CTCs compared to CK+EpCAM markers.

### Example 6: Diagnosing Patients

In this prophetic example, a kit is used to diagnose a patient for breast cancer. Blood is drawn from a patient. The blood is filtered and prepared using materials provided by the kit. The prepared blood sample is stained with antibodies provided by the kit to quantify the biomarkers described herein, and then the sample is imaged. Imaging results are analyzed (e.g., by a computer) to determine a diagnosis of breast cancer.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of identifying breast cancer in a subject, the method comprising:
a) providing a biological sample from the subject; and
b) determining a differential level of at least one biomarker of a biomarker set in the biological sample, wherein the differential level of the at least one biomarker of the biomarker set compared to a healthy control or a reference value is indicative for the presence of breast cancer in the subject, and wherein the biomarker set comprises CD55, LSR, MARCKSL1, GPC1, SLC9A3R1, CXADR, SHROOM3, MUC1, PPP1R16A, ATP1B1, RHOD, SYTL2, or any combination thereof.

2. The method of claim 1, wherein the biomarker set comprises CD55, LSR, MARCKSL1, GPC1, SLC9A3R1, CXADR, SHROOM3, MUC1, PPP1R16A, ATP1B1, RHOD, and SYTL2.

3. The method of claim 1 or claim 2, wherein a differential level of a biomarker selected from CD55, LSR, MARCKSL1, GPC1, SLC9A3R1, CXADR, SHROOM3, MUC1, PPP1R16A, ATP1B1, RHOD, or SYTL2 is a level higher than the healthy control or the reference value.

4. The method of claim 1 or claim 2, wherein a differential level of a biomarker selected from CD55, LSR, MARCKSL1, GPC1, SLC9A3R1, CXADR, SHROOM3, MUC1, PPP1R16A, ATP1B1, RHOD, or SYTL2 is a level lower than the healthy control or the reference value.

5. The method of any one of claims 1-4, wherein the method further comprises using RNA sequencing to obtain the differential level of the at least one biomarker.

6. The method of any one of claims 1-4, wherein the method further comprises using one or more antibodies to obtain the differential level of the at least one biomarker.

7. The method of claim 6, wherein the method further comprises imaging the biological sample.

8. The method of claim 6, wherein the method further comprises using western blot, ELISA, or mass spectrometry to obtain the differential level of the at least one biomarker.

9. The method of any one of claims 1-8, wherein the method further comprises morphological analysis of the biological sample.

10. The method of any one of claims 1-9, wherein the biological sample is a biofluid.

11. The method of claim 10, wherein the biofluid is blood, plasma, or serum.

12. The method of claim 10, wherein the biofluid is blood.

13. The method of any one of claims 1-12, wherein the sample is obtained prior to the onset of symptoms.

14. The method of any one of claims 1-12, wherein the sample is obtained after the onset of symptoms.

15. The method of any one of claims 1-14, wherein the subject is a human.

16. A kit for performing the method of any one of claims 1-15.

17. A method of identifying breast cancer in a subject, the method comprising:
a) providing a biological sample from the subject; and
b) determining a differential level of at least one biomarker of a biomarker set in the biological sample, wherein an increased level of the at least one biomarker of the biomarker set compared to a healthy control or a reference value is indicative for the presence of breast cancer in the subject, and wherein the biomarker set comprises MARCKSL1 and SLC9A3R1.

18. The method of claim 17, wherein the biomarker set further comprises ATP1B1 and RHOD.

19. The method of claim 17, wherein the biomarker set comprises MARCKSL1, ATP1B1, and SLC9A3R1.

20. The method of claim 17, wherein the biomarker set comprises MARCKSL1, RHOD, and SLC9A3R1.

21. The method of any one of claims 17-20, wherein the biomarker set further comprises LSR.

22. The method of any one of claims 17-21, wherein the biomarker set further comprises CD55, GPC1, CXADR, SHROOM3, MUC1, PPP1R16A, SYTL2, or a combination thereof.

23. The method of any one of claims 17-22, wherein the method further comprises using RNA sequencing to obtain the differential level of the at least one biomarker.

24. The method of any one of claims 17-22, wherein the method further comprises using one or more antibodies to obtain the differential level of the at least one biomarker.

25. The method of claim 24, wherein the method further comprises imaging the biological sample.

26. The method of claim 24, wherein the method further comprises using western blot, ELISA, or mass spectrometry to obtain the differential level of the at least one biomarker.

27. The method of any one of claims 17-26, wherein the method further comprises morphological analysis of the biological sample.

28. The method of any one of claims 17-27, wherein the biological sample is a biofluid.

29. The method of claim 28, wherein the biofluid is blood, plasma, or serum.

30. The method of claim 28, wherein the biofluid is blood.

31. The method of any one of claims 17-30, wherein the sample is obtained prior to the onset of symptoms.

32. The method of any one of claims 17-30, wherein the sample is obtained after the onset of symptoms.

33. The method of any one of claims 17-32, wherein the subject is a human.

34. A kit for performing the method of any one of claims 17-33.

35. A method of identifying breast cancer in a subject, the method comprising:
a) providing a biological sample from the subject; and
b) determining a differential level of at least one biomarker of a biomarker set in the biological sample, wherein an increased level of the at least one biomarker of the biomarker set compared to a healthy control or a reference value is indicative for the presence of breast cancer in the subject, and wherein the biomarker set comprises MARCKSL1, SLC9A3R1, and RHOD.

36. A method of identifying breast cancer in a subject, the method comprising:
a) providing a biological sample from the subject; and
b) determining a presence or an absence of at least one biomarker of a biomarker set in the biological sample, wherein the presence or the absence of the at least one biomarker of the biomarker set compared to a healthy control or a reference value is indicative for the presence of breast cancer in the subject, and wherein the biomarker set comprises CD55, LSR, MARCKSL1, GPC1, SLC9A3R1, CXADR, SHROOM3, MUC1, PPP1R16A, ATP1B1, RHOD, SYTL2, or a combination thereof.
